(19) United States
(12) Patent Application Publication
(10) Pub. No.: US 2026/0174505 A1
(43) Pub. Date: Jun. 25, 2026

(54) Electroporation with Immunotherapy to Generate a Pro-Inflammatory Response

(71) Applicant: RadioClash, Ltd. Co., HOUSTON, TX (US)

(72) Inventors: DUANE BESTER, HOUSTON, TX (US); Tricia Padich, HOUSTON, TX (US); Yunfei Qiao, Pearland, TX (US); Xu Huang, Pearland, TX (US)

(21) Appl. No.: 19/173,659

(22) Filed: Apr. 8, 2025

Related U.S. Application Data
(60) Provisional application No. 63/575,878, filed on Apr. 8, 2024.

(51) Int. Cl. A61B 34/20; A61B 17/32; A61B 17/34; A61B 18/00

(52) U.S. Cl. CPC A61B 34/20; A61B 17/320016; A61B 17/3423; A61B 2018/00613; A61B 2018/00839

(57) ABSTRACT

An improved electroporation probe for performing irreversible and reversible electroporation, and administering medicinal drugs at a target tissue is disclosed. An associated lumen of a trocar of the probe is connected to a source of injectable medicinal drug (such as an immunotherapy agent). A probe holder is operable to seal/unseal perforations associated with the trocar through a slidable tube covering the trocar. A plunger of the probe can be operated to administer desired doses of medicinal fluid through the lumen and associated perforations. In an embodiment, the trocar and an electrically conductive region on the tube are respectively connected to cathode and anode terminals of a DC battery. An electric field of desired strength is produced between trocar and the conductive region by sliding the tube to a corresponding position. The probe can be used perform electroporation and to administer desired dose of medicinal drug at the target tissue.

### BACKGROUND

Irreversible electroporation is a treatment modality used for a variety of diseases including but not limited to various types of cancers. A current flowing through two or more probes inserted within a tumor region generates a voltage differential and corresponding field strength. Permanent or temporary pores will be created within the cellular membranes of the tumor region in which the probes are inserted, depending on the electrical field strength.

A higher electrical field strength causes permanently open cellular membrane pores created by electroporation, leading to loss of homeostasis within the cells, and corresponding cell death. This type of electroporation is called irreversible electroporation. Reversible electroporation employs a lower electrical field strength, to cause cellular membrane pores to become temporarily open and eventually reseal. Thus, there is no loss of homeostasis, and no corresponding cell death. Reversible electroporation has been used for the treatment of a variety of diseases by allowing for the delivery of medications and materials into cells that are temporarily permeabilized, as well as allowing interaction between tumor antigens and inflammatory factors released from the permeabilized cell into the microenvironment, and interaction with solutions injected within the same region.

Probes inserted into a tumor region and programmed to a high electrical field strength can cause both irreversible electroporation to occur in regions closer in proximity to the probes, and reversible electroporation to occur in regions further away from the probes, due to differences in electrical field strength in relation to the location of the probes.

Enhanced electroporation therapy (sometimes called combination electroporation therapy) is a new treatment modality that utilizes a high electrical field strength to cause irreversible electroporation to cells in closer proximity to the probes, while simultaneously injecting a medication or material into the tumor region to allow for increased cellular uptake and microenvironmental interaction in tumor regions further away from the probes that have had their cellular membranes temporarily permeabilized via the reversible electroporation effect. Such microenvironmental interaction was observed in irreversible electroporation, where there was observed modulation of the stroma of the tumor microenvironment through increasing microvessel density and softening the extracellular matrix, leading to the recruitment of immune-activated cells into a tumor bed. See X. Gong et al. “Advances of Electroporation-Related Therapies and the Synergy with Immunotherapy in Cancer Treatment” Vaccines (Basel). 2022 November; 10(11): 1942.

Preclinical studies have found enhanced electroporation therapy to be more effective at inducing tumor cell death than either irreversible electroporation or intratumoral injection of chemotherapy alone in various types of human cancer cell lines including but not limited to liver cancer, pancreatic cancer, head and neck cancer, brain cancer, and secondary metastases; and utilizing a variety of materials including but not limited to various types of chemotherapy, immunotherapy, genetic material, contrast agents, and nanoparticles. The efficaciousness of the results from preclinical experiments suggests that enhanced electroporation therapy is a promising therapy for the treatment of various cancers and tumors.

Probes inserted into a tumor region or other tissue region (such as a draining lymph node) and programmed to a high electrical field strength can cause the release of local inflammatory factors, as well as antigens and epitopes from the region of tissue undergoing electroporation. The release of inflammatory factors activates the innate immune response, while the release of antigens and epitopes can lead to the sensitization of cells involved in the adaptive immune response, including but not limited to cytotoxic T cells and helper T cells. Sensitization of adaptive immune response cells to these released tissue antigens and epitopes has been shown to cause the maturation of adaptive immune response cells that target and destroy the tumors from which these antigens and epitopes are released.

Matured adaptive immune response cells can destroy both the local tumor, and also circulate throughout the body and target metastatic tumor and cancer cells that share antigens, epitopes, and damage-associated molecular patterns (DAMPs) with the original tumor that had undergone electroporation, in a phenomenon called the abscopal effect. This process is the basis of tumor vaccines. Additionally, when immunotherapies are injected either systemically or locally, the pro-inflammatory immune response is up-regulated, leading to an increase in tumor antigen presentation by innate immune cells, and subsequently an increase in the amount of circulating adaptive immune cells that are sensitized and targeted towards the antigens and epitopes released from the tumor regions that have undergone electroporation.

U.S. Pat. No. 11,660,139 (incorporated by reference) discusses use of enhanced or combination electroporation with administration of immunotherapy.

There exists a need for improved devices and methodologies to perform enhanced electroporation therapy with immunotherapy agent administration. U.S. Pat. No. 11,660,139 and U.S. patent application Ser. No. 18/515,326 (both incorporated by reference) disclose some preferred devices for combined electroporation and drug or immunotherapy administration. What is needed is a device for enhanced therapy, to use with protocols to effectively administer each of a variety of immunotherapy agents together with electroporation.

Furthermore, there exists a need for an improved electroporation device for simultaneous infusion of gel-like formulations (such as hydrogels and cryogels) which can be impregnated with various drugs and injected into tissues/organs. Such a device would allow the injected formulation to sit within the tissue/organ without being ejected through the needle tract (which is otherwise the path of least resistance for an injected fluid or suspension) which introduced the injectable into the target tissue/organ.

Still further, there also exists a need for an electroporation device which would be able to treat vascular malformations with both irreversible and reversible electroporation and select chemotherapies such as bleomycin.

### SUMMARY

A variety of electroporation devices can be used to combine electroporation and immunotherapy administration, including the device shown in U.S. patent application Ser. No. 18/515,326 (incorporated by reference), and in FIGS. 1A-E, 2A-B, 3A-D, 4A-D, 5A-B, and 6A-D and the specification at pp. 7-27 below, which includes:
a hollow tubular probe having a series of perforations towards the end of the probe, which ends in a sharp tip, where the opposite end of the probe is connected to a fluid injector;
a movable tubular sleeve covering the end of the probe which is moved to seal and unseal the perforations;
the end of the movable sleeve is connected to the terminal of a power source and the tip of the probe is connected the other terminal of the power source; and
the movable sleeve is connected to a probe holder having an interior channel, where the tubular movable sleeve extends through the interior channel, and having a sliding tab attached to the tubular sleeve wherein movement of the sliding tab moves the movable sleeve to either cover or uncover the perforations of the probe.

The sharp tip is used to pierce the flesh of the patient and allow access of the electroporation probe to the treatment site. The immunotherapy agent, in solution, is fed to the probe. An electric field is applied and the sleeve is moved back to uncover the perforations, and to allow manipulation of an external injector mechanism to provide the therapeutic solution at the treatment site.

Other suitable embodiments of a device for enhanced electroporation with immunotherapy administration are described in U.S. Pat. Nos. 11,660,139, 5,472,441, 7,160,296, and 6,071,280 (all incorporated by reference).

A preliminary step in enhanced electroporation therapy is usually to first Infuse saline, before electroporation. This increases safety of the therapy by decreasing the maximum temperature of the nonthermal ablation zone of the electroporation.

For cancers and tumors, the electroporation treatment site is generally directly into or near to the tumors, or for non-specific tumors (e.g., lymphomas and leukemias) directly into the lymph nodes.

Among the immunotherapy agents which can be administered by enhanced electroporation for treating cancers and tumors are:
non-specific immunotherapies and adjuvants, including: upregulating bacteria such as Bacillus Calmette-Guerin (BCG) and attenuated Staphylococcus aureus, attenuated viruses such as herpes simplex virus 1 (HSV-1), interleukin-2 (IL-2), interleukin-12 (IL-12), and interferon-alfa, interferon gamma, as well as other pro-inflammatory cytokines, oncolytic viruses, and antigen presenting cells;
pro-inflammatory immunomodulating drugs, including, thalidomide (for treating cancers and leprosy lesions), lenalidomide (REVLIMID) (for multiple myeloma, smoldering myeloma, and myelodysplastic syndromes);
pro-inflammatory cancer vaccines e.g. PROVENGE (sipuleucel-T);
immune checkpoint inhibitors including CTLA-4, PD-1 and PD-L1 (for diseases where the treatment objective is to upregulate the immune response, by downregulating anti-inflammatory regulatory T cells);
TLR3 agonists and TLR9 agonists;
anti-cancer agents like IMLYGIC® (talimogene laherparepvec) for melanoma;
CAR+ T cells (for a variety of tumors);
toll like receptor 9 ligand and toll like receptor 7 ligand (for diseases where the treatment objective is to upregulate the immune response); and
Durvalumab, Tremelimumab, Atezolizumab or any combination of Durvalumab, Tremelimumab, and Atezolizumab.

Enhanced electroporation with immunotherapy administration includes a number of variables which must be identified and set, for treatment with any one or any combination of the above immunotherapy agents. These variables include, for each disease or condition, setting the electric field strength (which varies depending on tissue type contacted, current and voltage from the power source, and the separation of the outputs from the anode and the cathode at the treatment site), length of time or frequency of application of the electric field, distance of the electrodes from a tumor or treatment site, interval between the electric field application and the administration of the immunotherapy agent, and the dosage and frequency of administration of the immunotherapy agent.

The electric field can be applied with direct current or with pulses. When using pulses, additional variables are pulse length, total number of pulses, pulse frequency (intervals between them), grouping of pulses, e.g., 1 to 10 groups of 10 pulses (10 clusters), pulse energy, and pulse amplitude. Other variables relate to pulse phasing.

### BRIEF DESCRIPTION OF FIGURES

FIG. 1A illustrates a first embodiment of the electroporation probe in a state of rest with the sliding tab in a released state and the movable sheath in the first position, sealing the perforations. Only the probe holder and the sliding tab of the electroporation probe are shown in cross-section.

FIG. 1B illustrates a magnified view of portion PQRS of the illustration of FIG. 1A. FIG. 1B also illustrates a proximal end of the solid trocar along with its corresponding electrical connection lead included within a tubular moveable sleeve and a polyimide tube.

FIG. 1C illustrates the electroporation probe of FIG. 1A with the sliding tab moved to unseal the perforations. Only the probe holder and the sliding tab of the electroporation probe are shown in cross-section.

FIG. 1D illustrates a magnified view of portion P′Q′R′S′ of the illustration of FIG. 1C. FIG. 1D also illustrates a proximal end of the solid trocar along with its corresponding electrical connection lead included within a tubular moveable sleeve and a polyimide tube.

FIG. 1E illustrates a cross-section of the movable sheath of the electroporation probe of FIGS. 1A and 1B.

FIG. 2A is a perspective view of distal end of shaft of FIG. 1A.

FIG. 2B is a cross-sectional view of distal end of the shaft of FIG. 2A taken along a plane passing through its longitudinal axis.

FIGS. 3A and 3B are elevational views of an exemplary tri-planar tip suitable for the tip of the trocar described herein.

FIG. 3C is a plan view of the tri-planar tip shown in FIGS. 3A and 3B.

FIG. 3D is a cross-sectional view of the tri-planar tip taken along plane AA′ illustrated in FIG. 3C.

FIG. 4A illustrates a second embodiment of the electroporation probe in a state of rest, with the sliding tab in a released state, and having the exemplary tri-planar tip of FIG. 3A-3D attached on the distal end of the movable stylet of the probe.

FIG. 4B illustrates an embodiment of the shaft of FIG. 4A with the sliding tab moved to unseal all the perforations the movable stylet.

FIG. 4C is a perspective view of distal end of shaft of FIG. 4A.

FIG. 4D is a perspective view of distal end of shaft of FIG. 4B.

FIG. 5A illustrates the third embodiment of the electroporation probe, where the probe holder and the sliding tab are shown in cross-section. The sliding tab is shown in a released state, covering the probe perforations.

FIG. 5B illustrates the electroporation probe of FIG. 5A with the sliding tab moved to uncover the probe perforations.

FIG. 6A is an elevational view of portion ABCD of FIG. 5A.

FIG. 6B shows the portion in FIG. 6A but wherein the movable sleeve is shown in cross-section.

FIG. 6C is an elevational view of portion EFGH of FIG. 5B.

FIG. 6D shows the portion EFGH in FIG. 6C but wherein the movable sleeve and the probe tip are shown in cross-section.

### DETAILED DESCRIPTION

The variables discussed in the Summary can be mathematically optimized manually, or using software that facilitates conventional physics-based user interfaces and couples systems of partial differential equations (such as COMSOL by Multiphysics). The first step in optimization is using animal models in routine experimentation to establish the data set, with extrapolation to humans, based on weight ratios. In optimization of a data set, one sets the objective, the variables and variable constraints.

In this case, the optimization objective is to maximize the absorption of the immunotherapeutic agent by the target cells in the test subject. A major variable to set is the immunotherapeutic agent dosage, which can be a constant, once set. The related variables, including dosage and frequency of administration, would be varied over limits, with a test of cellular absorption of the immunotherapeutic agent by the target cells conducted for each variation.

The constraints on the variables include safety-driven maximums for current and voltage, and maximums for electric field application time and frequency. Constraints on the distance of the electrodes from a tumor or treatment site would be set by finding electric field strength and determining reductions based on separation, then setting thresholds for maximum distance from the treatment site where the field strength drops below a threshold. Constraints on the maximum and minimum separation of the outputs from the anode and the cathode at the treatment site, are set by a maximum which reduces the electric field below a threshold, and the minimum set by safety concerns, like shorting. Other constraints would include length of time or frequency of application of the electric field, which could be defined based on known information about cellular absorption with electroporation.

When the electric field is applied in pulses, additional variables are pulse length, total number of pulses, pulse frequency (intervals between them), grouping of pulses, e.g., 1 to 10 groups of 10 pulses (10 clusters), pulse energy, pulse amplitude. Each of these variables would have reasonable constraints, consistent with maintaining a safe pulse energy over time and avoiding ablation.

Other variables relate to pulse phasing. The phasing could be, e.g., monophasic bipolar or monopolar biphasic, or other polarity and phasing combinations. The constraints on the phasing variables would have a broader range than the other variables on pulses, as phasing does not necessarily lead to higher energy imparted to tissues and increased risk of ablation—so safety is not a concern.

Examples of the optimization described above are set forth below.

In animal experiments, the efficaciousness of the treatment with a variety of variable settings can be accomplished by monitoring indicators of a pro-inflammatory immune response in the animals. The indicators monitored can be of one or more of: cytokines, fever, elevated white blood cells (WBCs), elevated heart rate or blood pressure, or elevation in any of: differential WBC count, neutrophil count, lymphocyte count, natural killer T cells and pro-inflammatory B cells. Another indicator can be down-regulation of T cells.

Ideally, the experiments are conducted with a multitude of variable settings, with several indicators determined at each different setting, where a specified combination of indicator changes (i.e., specified levels of indicator elevation or downregulation, as applicable) are scored as achieving the objective. As the variables are discrete, not continuous, linear programming can be selected as the optimization algorithm, and, for example, the Simplex algorithm can be the algorithm selected for the linear programming. See Wikipedia, “Simplex algorithm,” wikipedia.org.

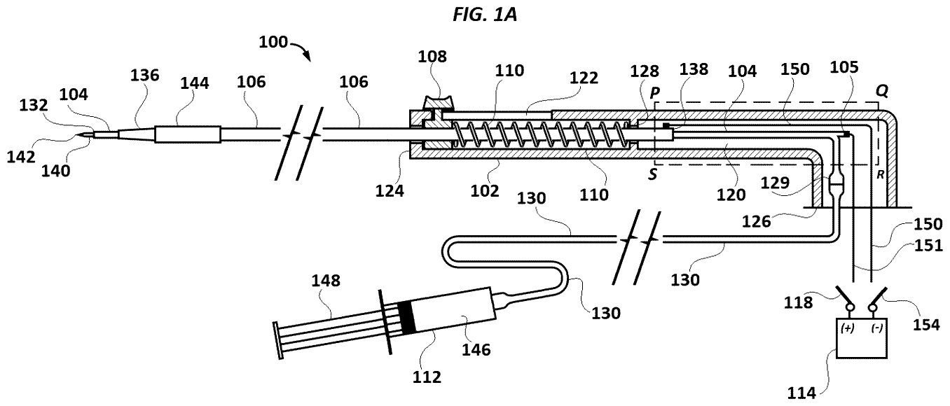

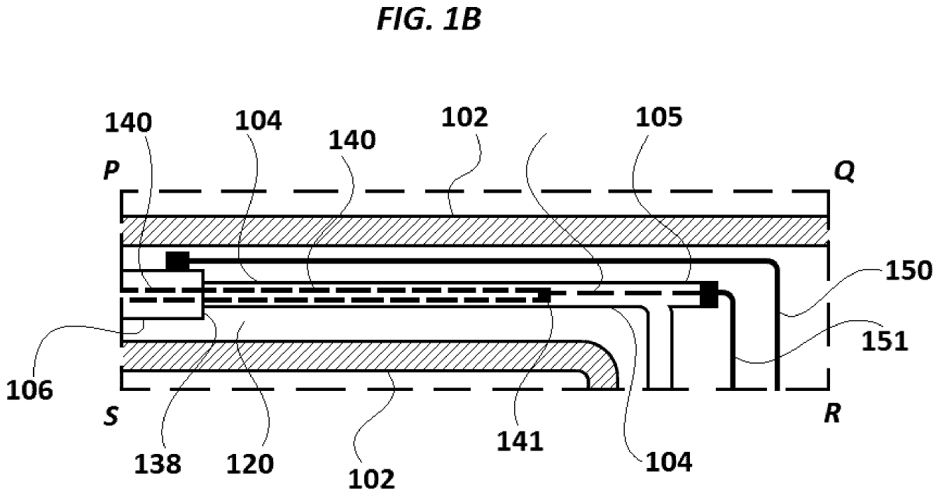

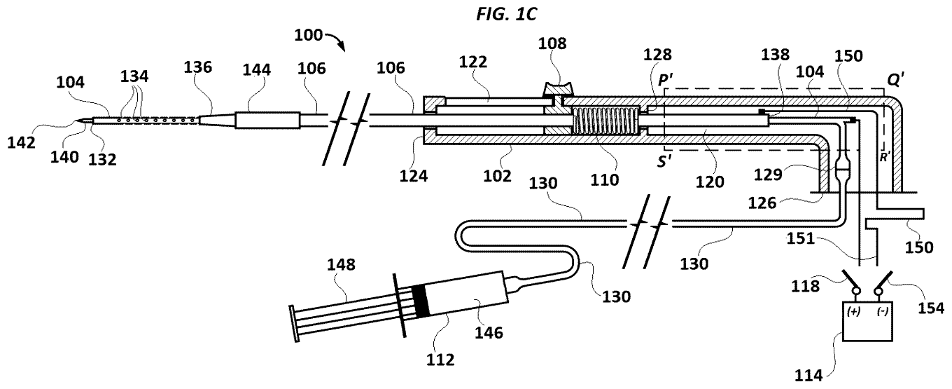

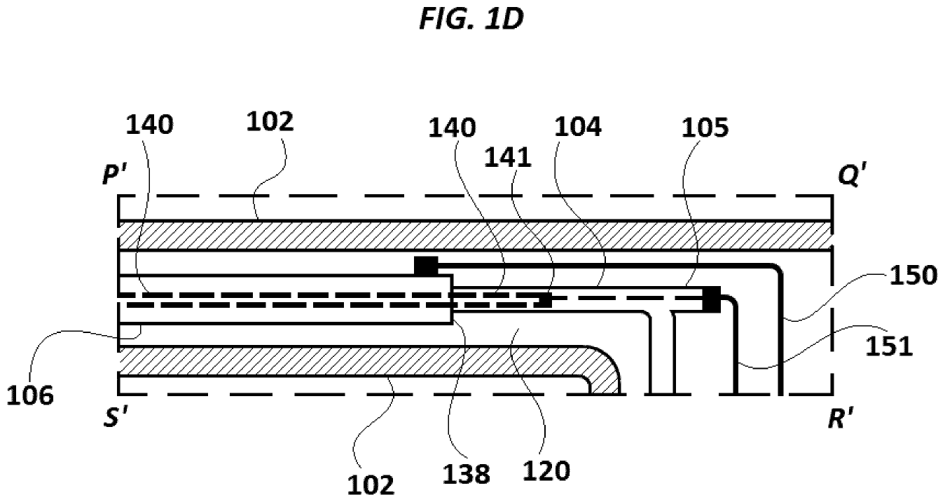

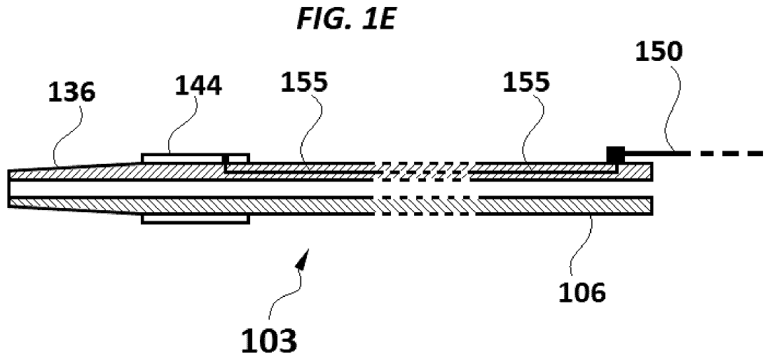

Reference will now be made in detail to a first embodiment of the electroporation probe of the present invention with reference to FIGS. 1A-E, 2A and 2B. As shown, the electroporation probe 100 incudes probe holder 102, a metallic shaft 140, a connector tube 130, a sliding tab 108, a compression spring 110, a fluid injector 112, a power source 114, an anode conductor lead 150 and a cathode connector lead 151. The shaft 140 is a solid trocar and is surrounded by a polyimide tube 104, a tubular movable sheath 106 and an anode portion 144.

The tubular movable sheath 106 is made of an electrical insulator material and extends through an interior channel 120 of the probe holder 102. The distal end portion of sheath 106 is in the form of a tapered insulator portion 136. Within the interior channel 120, tubular movable sheath 106 is surrounded by the compression spring 110 which is in an uncompressed (or a partially compressed) state when at rest as shown in FIG. 1A.

The sliding tab 108 is confined to slide to various predefined positions (not illustrated) within a longitudinal sliding slot 122 on the probe holder 102. When at rest, the sliding tab 108 is positioned adjacent the distal end 124 of probe holder 102 by the uncompressed (or partially compressed) spring 110. Movement of spring 110 towards proximal end 126 of probe holder 102 is restricted by an annular restriction 128 within the interior channel 120. Movement of sliding tab 108 away from the distal end 124 of probe holder 102 requires overcoming decompression force of the spring 110. The sliding tab 108 is attached to the tubular movable sheath 106. As a result, longitudinal movement of the sliding tab 108, towards restriction 128 or towards distal end 124 of probe holder 102 also moves the tubular movable sheath 106 in the same direction (See FIGS. 1A and 1B). In either situations, i.e. whether the sliding tab 108 lies adjacent to distal end 124 of probe holder 102, or whether the sliding tab 108 lies adjacent to annular restriction 128, the proximal end 138 of the movable sheath 106 remains within the interior channel 120 of the probe holder 102.

The polyimide in tube 104 is electrically conductive and extends through the tubular movable sheath 106, and sheath 106 is slidable over the polyimide tube 104. The electrical insulator portion 136 at the distal end of the tubular movable sheath 106 tapers over the polyimide tube 104. When the electroporation probe device 100 is in resting state, the distal end 132 of the polyimide tube 104 extends beyond the insulator portion 136 of the tubular movable sheath 106 and the proximal end 129 of the polyimide tube 104 extends beyond the proximal end 138 of the tubular movable sheath 106. A portion of length of the polyimide tube 104, towards its distal end 132, includes multiple longitudinal arrays of perforations 134 which access inner lumen of the polyimide tube 104 (see FIG. 1C).

Longitudinal movement of the tubular movable sheath 106 over the polyimide tube caused by longitudinal displacement of sliding tab 108 also causes sealing and unsealing of perforations 134. When the sliding tab 108 lies adjacent to the distal end 124 of probe holder 102 all perforations 134 are covered and sealed by the tubular movable sheath 106, and when the sliding tab 108 lies adjacent to restriction 128 all perforations 134 get uncovered and unsealed.

The proximal end 129 of the polyimide tube 104 is connected to the fluid injector 112 through the connector tube 130. The fluid injector 112 further includes a fluid reservoir 146 and a plunger 148. Based on requirements, a suitable medicinal fluid (including a medicinal drug such as immunotherapy agent, an anti-cancer agent, a cancer therapeutic, biological cells, and/or saline) is stored in the fluid reservoir 146.

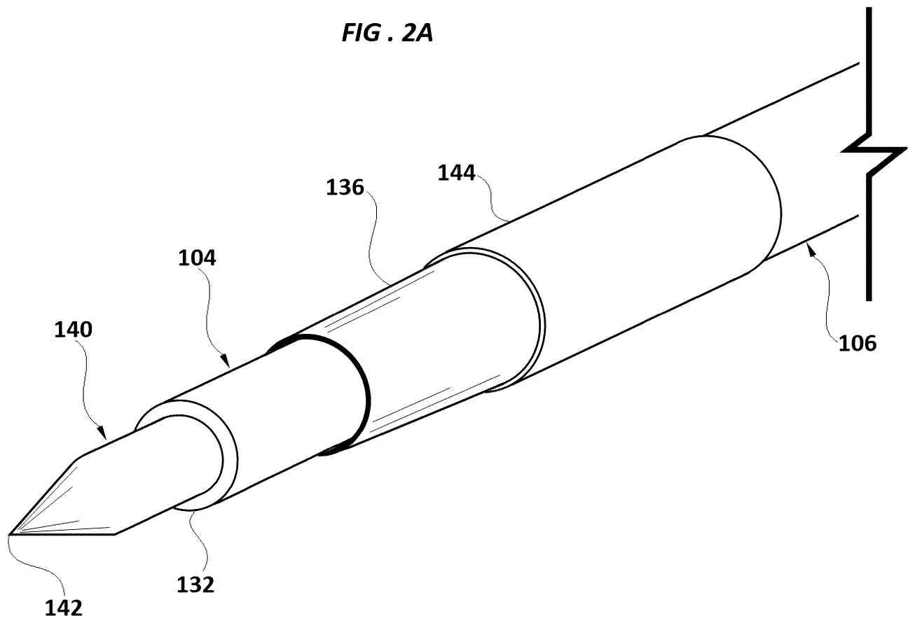

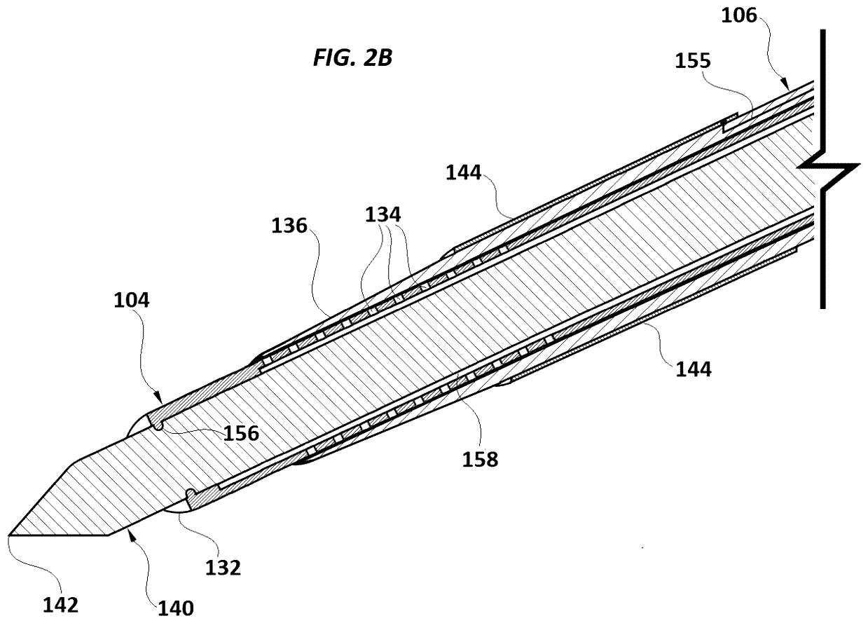

The solid trocar 140 is an electrically conductive, and preferably, a single elongated metallic rod (more preferably, it is a metal alloy suitable for application requirements and having good electrical conductivity). The solid trocar 140 extends through the inner lumen of the polyimide tube 104 (see FIGS. 1A-D). The diameter of the inner surface of the polyimide tube 104 is greater than the diameter of the outer surface of the solid trocar 140. As a result, a gap or a fluid passage 158 (see FIG. 2B) exists between the inner surface of the polyimide tube 104 and the outer surface of the solid trocar 140 (see FIG. 2B). During operation of the electroporation probe device 100 this gap, which is fluid passage 158, provides a flow space for the medicinal fluid being injected into the polyimide tube 104 by the fluid injector 112 through the connector tube 130. All perforations 134 have access to the fluid passage 158.

Distal end 142 (hereinafter referred to as tip 142) of the solid trocar 140 extends beyond the distal end 132 of the polyimide tube 104, and is preferably tapered. Still further, the distal end 132 of the polyimide tube 104 is preferably attached in a leakproof manner with the solid trocar 140 through an annular click seal 156 (see FIG. 2B) which fits onto the solid trocar 140. The proximal end 141 of the solid trocar 140 remains covered under the polyimide tube 104.

The diameter of the cathode conductor lead 151 is smaller than the diameter of solid trocar 140.

The proximal end 141 of the solid trocar 140 along with the polyimide tube 104 is connected to the cathode terminal of a DC power source (a DC battery) 114 through the conductor lead 151.

The conductor lead 151 enters the polyimide tube 104 through a small leakproof tubular branch 105. The entire length of the anode conductor lead 151, including connection points with the proximal end 141 of the solid trocar 140, tubular branch 105 and polyimide tube 104, and the power source 114, is covered with an electrical insulator material.

The outer surface of a longitudinal portion of the movable sheath 106, adjacent to its tapered insulator portion 136, is covered by a layer of electrically conductive material to form the anode portion 144. The length of the anode portion 144 is greater than the length of the tapered insulator portion 136 of sheath 106. Preferably, for a 17-gauge probe, the length of the anode portion is less than or equal to 15 mm. The movable sheath 106 further includes an embedded conductor lead 155 (see FIGS. 1E and 2B). The embedded conductor lead 155 is embedded longitudinally within the insulator wall of the movable sheath 106 and electrically connects the anode portion 144 with one end of the anode conductor lead 150 lying on the proximal end 138 of the movable sheath 106. The other end of the anode conductor lead 150 is connected to the anode terminal of the power source 114. So the anode portion 144 is connected to the anode terminal of the power source 114 through the embedded conductor lead 155 and through the anode conductor lead 150. The entire length of the anode conductor lead 150, including the connection points with the proximal end 138 and with the power source 114, is covered with an electrical insulator material. While the conductor leads 150 include an electrical switch 154 for selectively connecting it with the anode terminal of the power source 114, the conductor leads 151 include an electrical switch 118 for selectively connecting it with the cathode terminal of the power source 114.

Sliding of movable sheath 106 over the polyimide tube 104 causes covering and uncovering of perforations 134. While those perforations 134 which get covered under the tubular movable sheath 106 get sealed, those which become uncovered (or are not covered) by the tubular movable sheath 106 become (or remain) unsealed. Hence, an operator of the electroporation probe device 100 can cause sealing (or unsealing) of desired number of perforations 134 by moving the sliding tab 108 longitudinally within slot 122, and cause the tubular movable sheath 106 to slide to a desired position over the polyimide tube 104. When the electroporation probe device 100 is at rest, the tip 142 and a distal portion of the polyimide tube 104 (lying adjacent to the tip 142 and lying adjacent to the tapered insulator portion 136), having no perforations 134, remain exposed and uncovered by the tubular movable sheath 106, and all perforations 134 lie covered and sealed by the tubular movable sheath 106.

In the first embodiment of the invention, the preferred length of the tapered electrical insulator portion 136 at the distal end of the tubular movable sheath 106 is about 7.5 mm-1 cm. This minimizes the risk of electrical arching at operating voltages. In other embodiments of the invention, different gauge probes may have different dimensions for the tapered electrical insulator portion 136.

In the first embodiment of the invention, when the electroporation probe device 100 is at rest, the preferred length of the exposed, unsealed portion of the tube 104, when the sheath is in the first position, is between 15-25 mm, and more preferably, between 15-20 mm.

In the first embodiment of probe device 100, though the insulator portion 136 of the tubular movable sheath 106 is designed to be tapered towards the distal end, in other embodiments the insulator portion 136 can be non-tapered and the channel for fluid between the distal end of insulator portion 136 and trocar 140 can be sealed with other means.

Next, in the first embodiment, when the electroporation probe device 100 is at rest, movement of sliding tab 108 away from the distal end 124 of probe holder 102 requires overcoming decompression force of the spring 110. On application of sufficient force by the operator, the sliding tab 108 (along with the movable sheath 106 attached to it) can be moved towards annular restriction 128, thereby compressing spring 110 (see FIG. 1B). As movable sheath 106 slides along with sliding tab 108, its tapered insulator portion 136 moves away from tip 142 of solid trocar 140 and away from the exposed distal portion of the polyimide tube 104 (which lies adjacent to the tip 142), thus uncovering (and unsealing) perforations 134, previously hidden under the movable sheath 106. When the sliding tab 108 is placed next to the proximal end of slot 122, all perforations 134 get uncovered. In this position of the sheath 106, separation between the distal end 132 of the polyimide tube 104 and the distal end of said anode portion 144 is 27.5 to 30 mm.

The sliding slot 122 may include longitudinally distributed and equidistant restrictive notches (not illustrated) which would help the operator to station the sliding tab 108 at a desired position. In such an embodiment, moving the sliding tab towards the proximal end 126 of the probe device 100 would require force to overcome decompression force from spring 110 and to overcome frictional forces of respective notche/s. Similarly, moving the sliding tab towards the proximal end 124 of the probe device 100 would require force to overcome frictional forces of respective notch/s.

Operation of electroporation probe device 100 for treating a target tissue site will now be explained in detail. In the first step, the distal end of probe device 100 (including tip of solid trocar 140) is inserted into the tissue to reach a target location. Thereafter, the sliding tab 108 is slid towards annular restriction 128 to slide the movable sheath 106 and to uncover and unseal a desired number of perforations 134. In the next step, using plunger 148, the medicinal fluid in reservoir 146 is injected into the polyimide tube 104 through the connector tube 130. The injected medicinal fluid travels through the gap or a fluid passage 158 between the inner surface of the polyimide tube 104 and the outer surface of the solid trocar 140 to be released through the unsealed perforations 134 for application at the target tissue site. Thereafter, switches 118 and 154 are turned on to power the solid trocar 140, the polyimide tube 104 and the anode portion 144. Because of the separation provided by the insulator portion 136 between the exposed portion of solid trocar 140, the exposed polyimide tube 104, and the anode portion 144, an electric field of desired strength is generated at the target tissue at lower current and electric field densities. Hence, the structure of electroporation probe device 100 achieves larger volumes of the treated tissue at lower current and electric field densities.

The strength of generated electric field can be varied either by varying separation between the exposed portion of solid trocar 140 and the anode portion 144, or by varying the voltage potential difference between the anode and cathode terminals of the power source 114.

It is to be understood that the relative dimensions (including lengths and gauge) of the solid trocar 140, the polyimide tube 104, the movable sheath 106, spring 110 and that of the longitudinal slot 122 for the sliding tab 108 may be selected based on treatment requirements.

Though in the first embodiment describes above, a DC battery has been used as a power source 114, other embodiments of the invention can use DC generators or other sources of DC supply as a power source. All such embodiments are within the scope of the present invention.

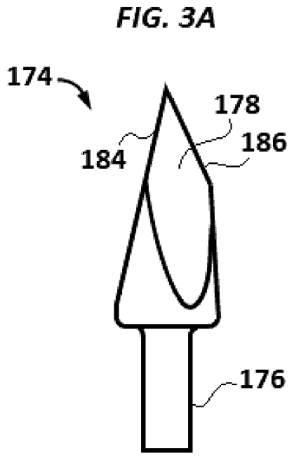

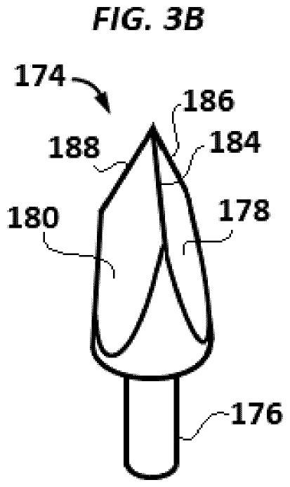

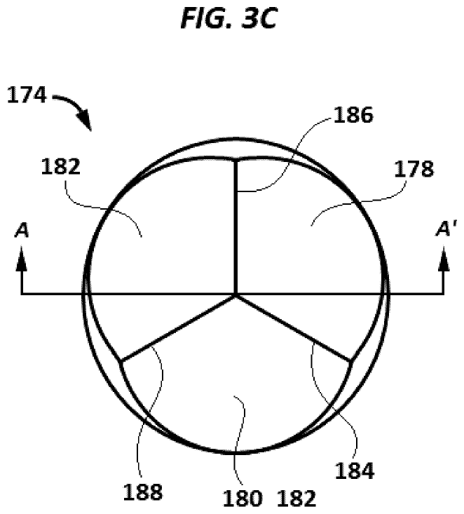

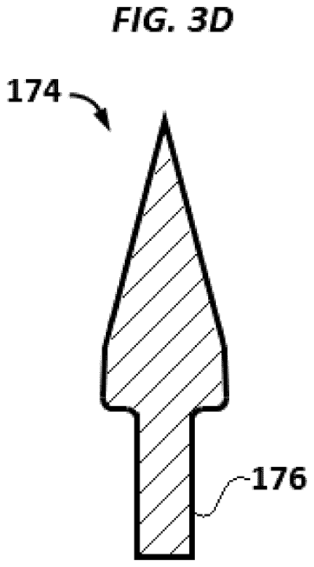

In other embodiments of the invention, modifications in the structure of the front tip 142 can be made. One such modified front tip is illustrated in FIG. 3A-3D. As shown, a modified tip 174 (preferably made of a metal alloy) includes a base shaft 176 and has a tri-planar tip surface. Its periphery includes three surrounding planes 178, 180 and 182. The interplanar edges 184, 186, and 188 are milled sharp for assisting the modified tip 174 to better push through tissues or cells.

Apart from one or more longitudinal arrays of perforations 134 on the polyimide tube 104, as described in the first embodiment, other embodiments of the present invention, based on delivery requirements of the medicinal fluid, may include other array structures or distribution patterns of perforations 134. Similarly, the dimensions and shape of perforations 134 in embodiments of the invention can also be selected as per delivery requirements of the medicinal fluid. All such modifications are within the scope of the present invention.

It is to be noted that medicinal fluid can be delivered to the target site either in the presence or absence of applied electric field. Though the first embodiment of the invention mentions application of the electric field after the delivery of medicinal fluid, it is to be noted that, as and when required, the order of delivery of medicinal fluid and application of electric field can also be reversed.

Electroporation probe 100 can be operated to treat vascular malformations with both irreversible and reversible electroporation, using select chemotherapies such as bleomycin.

Furthermore, the electroporation probe device 100 can be used for infusion of gel-like formulations (such as hydrogels and cryogels) which can be impregnated with various drugs and injected into tissues/organs. The electroporation probe device 100 allows the injectable to sit within the tissue/organ without being ejected through the probe path (which is otherwise the path of least resistance for an injected fluid).

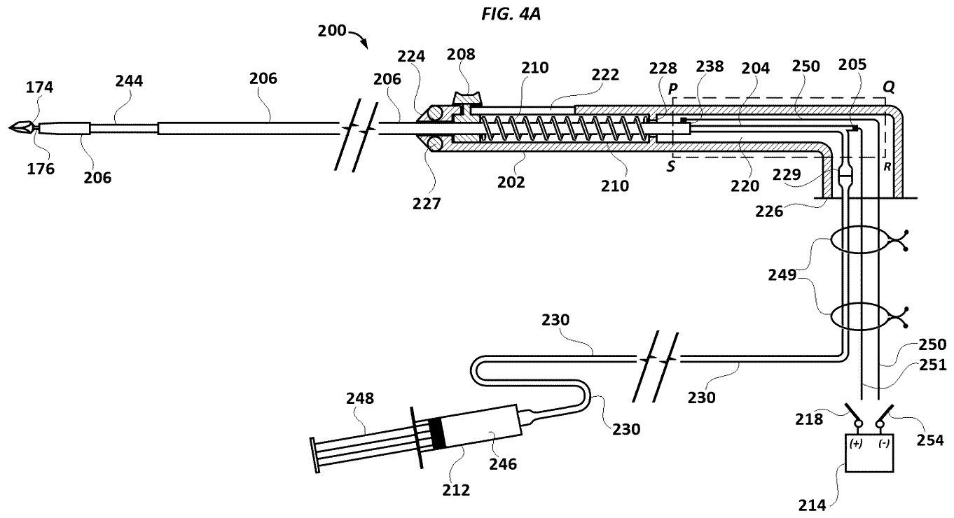

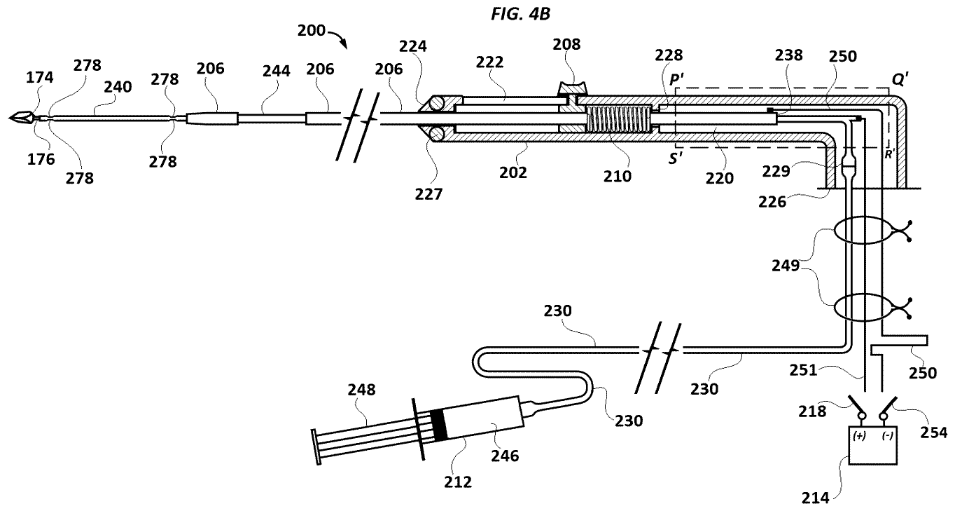

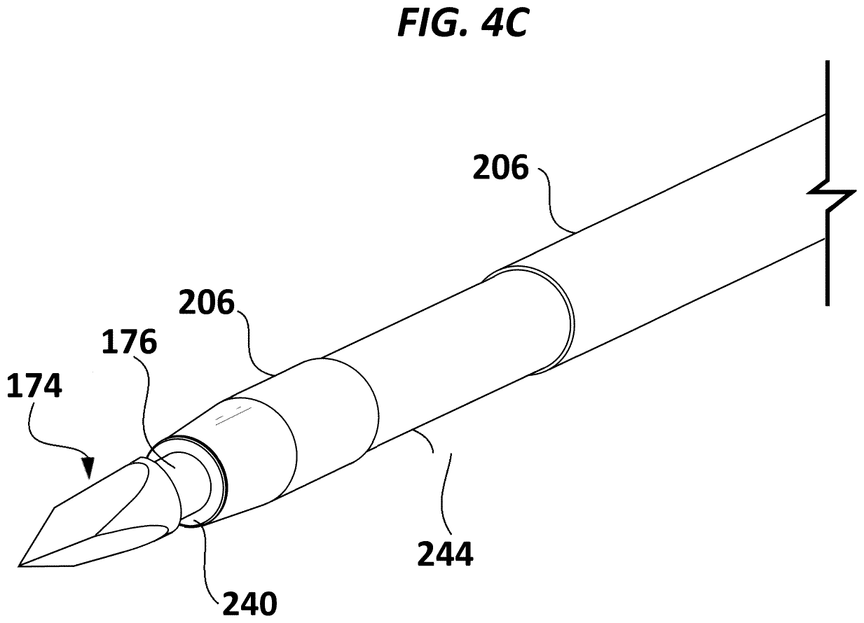

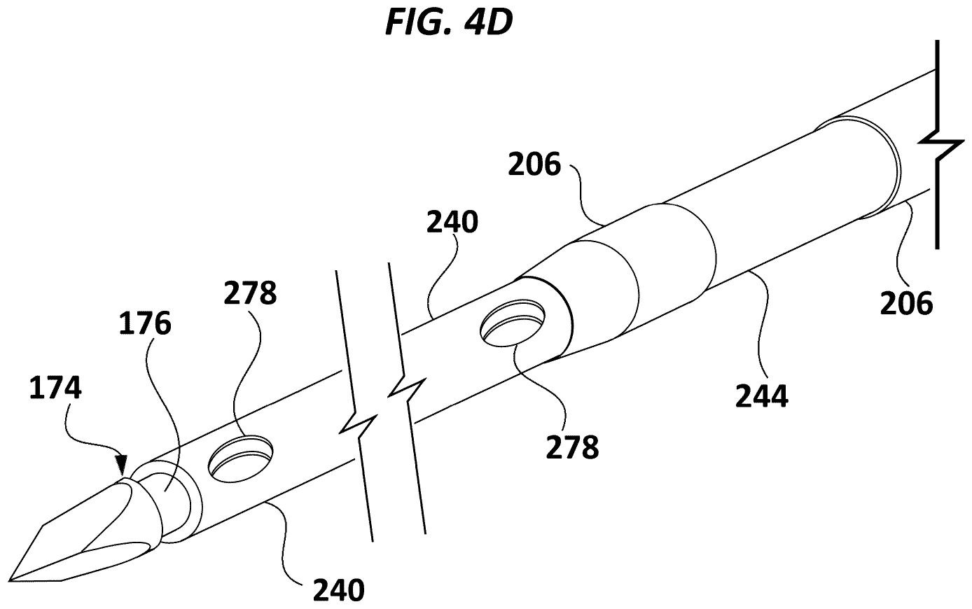

A second embodiment of the electroporation device in accordance with the present invention will now be described with reference to FIGS. 4A-4D. As can be seen in FIGS. 4A and 4B, the second embodiment of the electroporation device is a modification of the first embodiment. The entire structure, components and functioning of probe 200 is similar to that of probe device 100 except as described below.

In contrast to probe device 100, probe 200 includes binding clips 249 which assist in keeping lead 250, lead 251 and connector tube 230 (which correspond to lead 150, lead 151 and connector tube 130 of probe device 100) collectively in order and enhance better operability of the probe 200. Further, in contrast to the distal end 124 of probe device 100, the distal end 224 of probe 200 is tapered and includes an O-ring leak seal 227. During the operation of the tool, the leak seal 227 prevents fluids from entering the probe 200 from the distal end 224. Still further, in contrast to moveable solid trocar 140 of probe device 100, the probe device 200 includes an entirely different shaft 240. The shaft 240 is in the form of a hollow tubular metallic trocar. The trocar 240 is an electrically conductive, and preferably, a single elongated metallic tube (more preferably, it is made of a metal alloy suitable for application requirements and having good electrical conductivity). The shaft 240 is surrounded by a tubular movable sheath 206 made of an electrically insulator material. Towards its distal end, the tubular movable sheath 206 includes an electrically conductive anode portion 244. Separation between the distal end of the sheath 206 and the distal end of said anode portion 244 is_7.5-10 mm. The shaft (or the tubular trocar) 240 further includes two pairs of oppositely oriented fluid delivery perforations 278 and a trocar tip 176. The trocar tip 176 is similar to one described above and illustrated in FIGS. 3A-3D.

Instead of multiple longitudinal arrays of perforations 134 as in probe device 100, the solid trocar 240 includes only two pairs of oppositely oriented fluid delivery perforations 278. In its state of rest, only the trocar tip 176 remains exposed by the tubular movable sheath 206. Similarly when the sliding tab 208 is moved completely towards the proximal end of probe device 200 (as shown in FIG. 4B), the tubular movable sheath 206 is pulled proximally, exposing a longer length of trocar 240. In this state, along with the trocar tip 176, both pairs of opposite fluid delivery perforations 278 get exposed (and unsealed by the tubular movable sheath 206). FIGS. 4C and 4D respectively illustrate the distal end portion of shaft 240 in a state of rest, and when the sliding tab 208 moved completely towards the proximal end of probe device 200.

The length of the anode portion 244 is greater than the length of the portion of sheath 206 lying between the distal end of anode portion 244 and distal end of sheath 206.

During operation of the probe device 200, in either of the states, medicinal fluid, if needed to be delivered at a target site, is delivered by the unsealed pair/s of the opposite fluid delivery perforations 278. The overall operation of probe device 200 is similar to operation of probe device 100, as explained above.

Still further, electroporation probe 200 can be operated to treat vascular malformations with both irreversible and reversible electroporation, and select chemotherapies such as bleomycin.

Furthermore, electroporation probe 200 can be used for infusion of gel-like formulations (such as hydrogels and cryogels) which can be impregnated with various drugs and injected into tissues/organs. The electroporation probe device 200 allows the injectable to sit within the tissue/organ without being ejected through the shaft 240 path which introduced the injectable into the target tissue/organ.

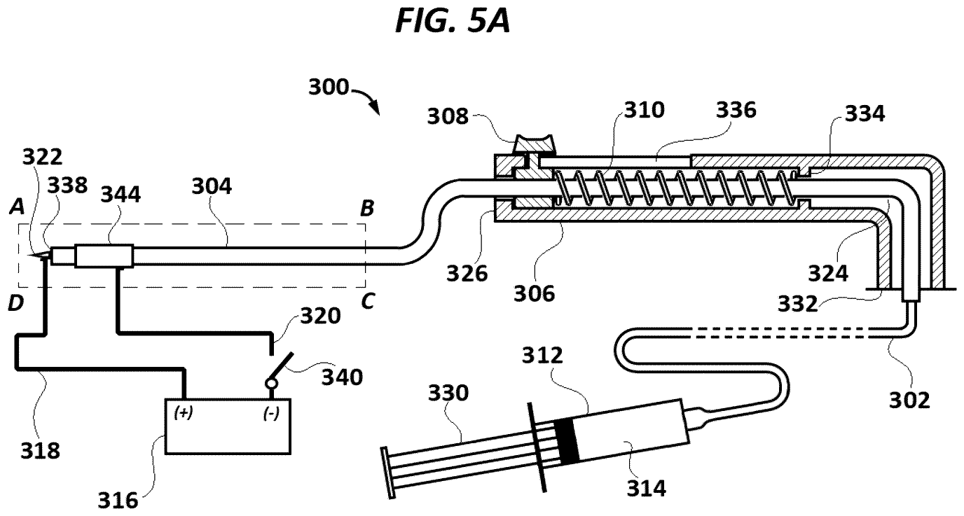

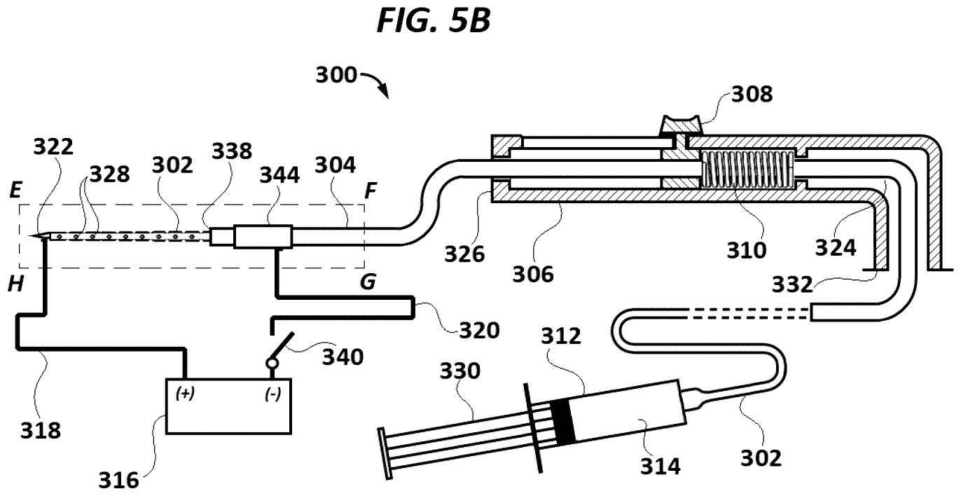

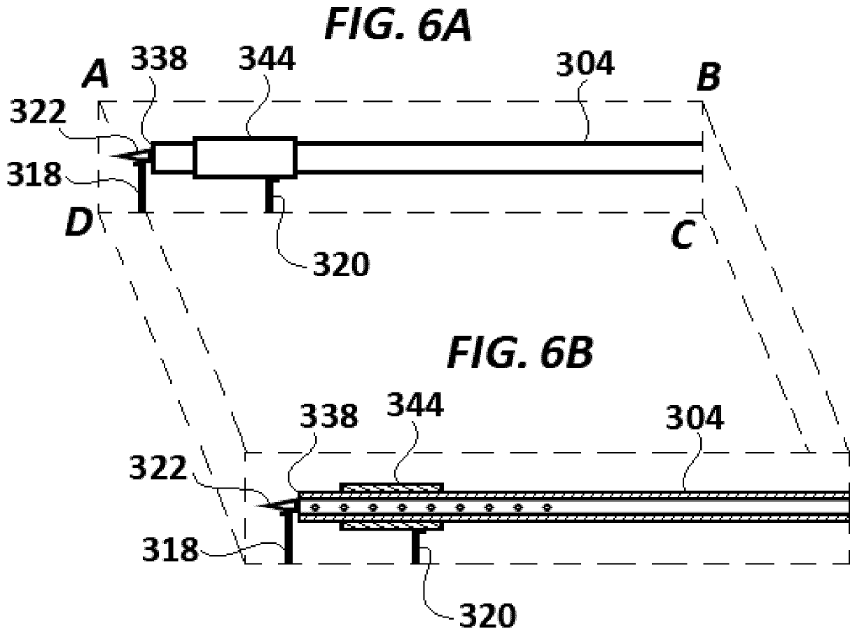

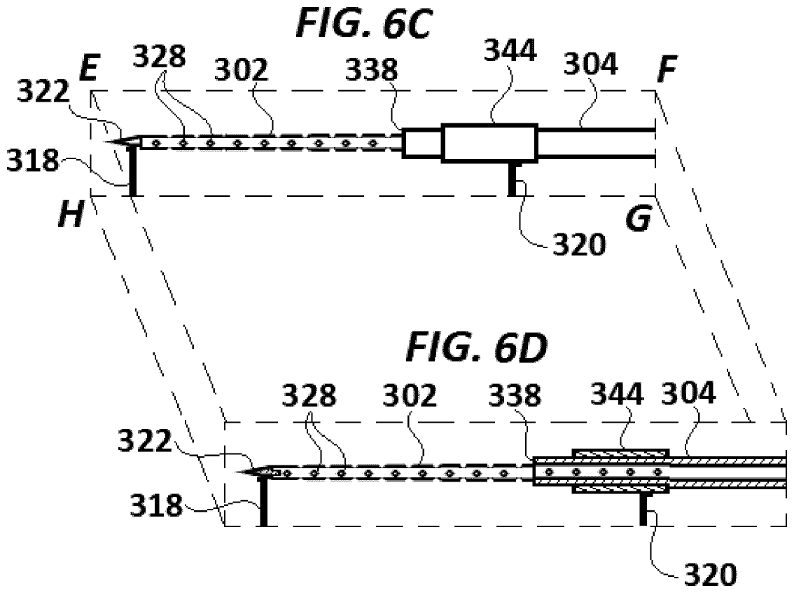

A third embodiment of the electroporation device will now be described with reference to FIGS. 5A and 5B. As shown, the electroporation device 300 includes a flexible hollow tubular flexible shaft 302, a flexible tubular movable sheath 304 covering a portion of length of shaft 302, a probe holder 306, a sliding tab 308, a compression spring 310, a fluid injector 312, a power source 316, cathode connection lead 318, and an anode connection lead 320. The flexible tubular movable sheath 304 is made of an electrical insulator material. Towards its distal end 338, a portion of the movable sheath 304 is covered by a layer of electrically conductive material to form the anode portion 344. The distance between the distal end 338 of the sheath 304 and the distal end of said anode portion 344 is_7.5-10_mm. The length of the anode portion 344 is greater than the distance between the distal end 338 of the movable sheath 304 and the distal end of the anode portion 344 (i.e. the end of the anode portion 344 lying towards distal end 338 of the movable sheath 304). The distal end of shaft 302 includes a pointed tapered tip 322. Tip 322 is metallic and its structure is similar to tip 174 described above and as illustrated in FIGS. 3A-D. The tip 322 is electrically conductive, and preferably, made of a metal alloy suitable for application requirements and having good electrical conductivity. A preset length of the shaft 302, towards its distal end (near the tip 322), includes multiple perforations 328 (see FIGS. 5B and 6B-D). The proximal end of shaft 302 is connected to the fluid injector 312 (preferably, a fluid syringe). The fluid injector 312 further includes a fluid reservoir 314 and a plunger 330.

Based on requirements, a suitable medicinal fluid (including a medicinal drug such as immunotherapy agent, an anti-cancer agent, a cancer therapeutic, biological cells, and/or saline) is stored in the fluid reservoir 314.

When electroporation device 300 is in resting state (as shown in FIG. 5A), shaft 302 extends through a channel 324 of probe holder 306 and a substantial length of shaft 302 from its distal end towards the proximal end is covered by movable sheath 304. In this state, perforations 322 remain covered and sealed by movable sheath 304. While distal end 338 of movable sheath 304 remains flush with the distal end of shaft 302 (and covers perforations 328), its proximal end extends into channel 324 of probe holder 306. Within probe holder 306, movable sheath 304 (including shaft 302 within) is surrounded by compression spring 310 (which is in an uncompressed state at rest).

Sliding tab 308 which is confined to slide within a longitudinal space 336 on the probe holder 306, is held against the distal end 326 of probe holder 306 by uncompressed spring 310. Movement of spring 310 towards proximal end 332 is restricted by an annular restriction 334 of the channel 324 within the probe holder 306. Sliding tab 308 is attached to movable sheath 304. As a result, longitudinal movement of the sliding tab 308 moves movable sheath 304 in the same direction.

Movement of sliding tab 308 away from the distal end 326 requires overcoming the decompression force of the spring 310. On application of force by the user, sliding tab 308 (along with the movable sheath 304 attached to it) is moved towards annular restriction 334, thereby compressing spring 310 (see FIG. 5B). As movable sheath 304 slides along with sliding tab 308, its distal end 338 moves away from tip 322, to expose and unseal the perforated portion of shaft 302. On removal of force on sliding tab 308, sliding tab 308 is pushed back towards distal end 326 of probe holder 306 by spring 310. Sliding tab 308 ends up at rest next to distal end 326, and movable sheath 304 also moves to cover (and seal) perforations 328 of shaft 302, and its distal end 338 ends up flush with tip 322.

The proximal end of tubular shaft 302 is connected to fluid injector 312. When movable sheath 304 is slid to expose (and unseal) perforations 328, any fluid carrying medication is injected through the injector 312, travels through tubular shaft 302, and is ejected from perforations 328.

The tip 322 of the shaft 302 is connected to a cathode terminal of a power source 316 (for example, a DC re-chargeable battery) through a flexible and insulated cathode connection lead 318. The anode portion 344 on the movable sheath 304 is connected to the anode terminal of the power source 316 through a flexible and insulated anode connection lead 320, including a connection switch 340. The closing (or opening) of switch 340 results in connection (or disconnection) of the anode portion 344 from the power source 316.

In other embodiments of the invention, a similar switch may be provided in the cathode connection lead 318 for connecting (or disconnecting) it from the power source 316. All such embodiments are within the scope of the present invention.

When Switch 340 is closed (i.e., in a circuit make position), an electric field is generated between the tip 322 and the anode portion 344 on the movable sheath 304. The strength of the electric field depends on the electric potential difference between the anode and cathode terminals of the power source and the amount of separation between the tip 322 and the anode portion 344.

The lengths of the shaft 302 as exposed by sliding the movable sheath 304 may be selected based on treatment requirements.

Though in the FIGS. 6A and 6B, the flexible and insulated cathode connection lead 318 is illustrated as lying exterior to shaft 302, in other embodiments of the invention it may lie within the hollow of probe 306 and may lie within the tubular body of shaft 302 and similarly remain connected to tip 322 from within.

Similarly, the flexible and insulated anode connection lead 320 is illustrated as lying exterior to the movable sheath 304, but in other embodiments it may lie within the hollow of movable sheath 304 (in parallel and exterior to the shaft 302) and remain connected to the anode portion 344 by extending across the walls of the sheath 304. Still further, in still other embodiments of the invention, the anode connection lead 320 may lie embedded within the walls of movable sheath 304 and be connected to the anode portion 344 from within. In all such embodiments, the anode connection lead 320 and the cathode connection lead 318 should not hinder smooth sliding of the movable sheath 304 over shaft 302.

Electroporation device 300 is for treating target cells by delivering cells or other biologics, or medications, with electroporation. For treating target cells or tissues, probe 306 is driven into the patient's body through an opening until tip 322 is placed at the target site. In the next step, depending on the amount of electroporation required, a desired strength of electric field is set between tip 322 plus exposed portion of the conductive cathode shaft 302 and the anode portion 344 of movable sheath 304. This is achieved by sliding the sliding tab 308 by a certain distance towards annular restriction 334, so that movable sheath 304 is moved relative to tip 322 and is placed at a desired separation with the tip 322. Thereafter, switch 340 is closed to generate an electric field between tip 322 plus exposed portion of the conductive cathode shaft 302 and the anode portion 344.

Before or during the treatment, based on requirements, the strength of electric field may be controlled (i.e., increased or decreased) by maneuvering sliding tab 308 to control the separation between the tip 322 and the anode portion 344.

In current embodiment, though the cathode connection lead 318 is connected to the tip 322 of the probe, it is to be noted that in other possible embodiments of the invention, instead being connected to the tip 322, the cathode connection lead 318 may be connected to any other portion or point on the entire length of the flexible shaft 302. Note that, in such embodiments of the invention, the shaft 302 would necessarily be made of an electrically conductive material. All such modifications in probe 300 are fully covered within the scope of the present invention.

Medicinal fluid (including a medicinal drug such as immunotherapy agent, an anti-cancer agent or biological cells) can be delivered to the target site either in the presence or absence of applied electric field. When an electric field is applied, if the separation between the tip 322 and the anode portion 344 on the movable sheath 304 has sufficient perforations 328 exposed and unsealed, medications, cells or biologics can be delivered to the target site by injecting them into shaft 302 using injector 312 (by pressing the plunger 330), until a desired quantity is ejected from the exposed perforations 328. However, if the number of exposed perforations 328 are insufficient to deliver the desired quantity, the separation between tip 322 and distal end 338 of the movable sheath 304 can be increased to unseal more perforations 328. As a result of an increase in separation, the applied electric field strength may also get reduced. The position may be changed after injection to again apply an increased strength electric field. Or, to deliver medications, cells or biologics to the target site in the absence of applied electric field, the switch 340 is turned off. Sliding tab 308 is moved to expose a desired length of the perforated portion of probe 306 before injection of medication, cells or other biologics.

Still further, It is to be noted that electroporation probe 300 can be operated to treat vascular malformations with both irreversible and reversible electroporation, and select chemotherapies such as bleomycin.

Furthermore, the electroporation probe device 300 can be used for infusion of gel-like formulations (such as hydrogels and cryogels) which can be impregnated with various drugs and injected into tissues/organs. The electroporation probe device 300 allows the injectable to sit within the tissue/organ without being ejected through the probe path which introduced the injectable into the target tissue/organ.

It is to be noted that, though in the embodiments described above, a DC battery has been used as a DC power source, in other embodiments of the invention, apart from a DC battery, the DC power source could well be a DC output generated from an AC supply through appropriate DC conversion circuits.

The following examples illustrate the protocols in animal models.

### Example 1: Treating Pancreatic Head Malignant Lesions

### Animal Model

A laparotomy is performed with direct visualization of the pancreatic head malignant lesion. The RadioClash Electroporation Device (R.E.D.) is the electroporation device described in FIGS. 1A-E, 2A-B and 3A-D and the description above at pp. 7-10, or the one in FIGS. 4A-B, 5A-D, 6A-B, 7 and 8 and the specification at pp. 10-13, though, any other suitable combination electroporation device can be used. The R.E.D. is connected to an electroporation generator. To eliminate dead air space in the inner cannula, which is in communication with the fluid infusion port and tubing at the proximal end of the device, a syringe containing toll-like receptor 9 (immunotherapy) solution is attached to the luer lock of the fluid infusion port and tubing, and flushed forward until solution is seen dripping from perforations at the distal portion of the inner cannula. The probe is now primed.

The probe is inserted into the pancreatic head malignant lesion under direct visualization. The outer sheath of the R.E.D. is unsheathed so that the anode on the tip of the outer sheath is 1 cm apart from the cathode at the tip of the inner cannula needle along a linear plane. The perforations along the entire length of the unsheathed inner cannula are exposed and can deliver the immunotherapy solution to the lesion. The generator is programmed to deliver 100 pulses each at a voltage of 1500V (which corresponds to a field strength of 1500V/cm, as the distance between the anode-cathode is 1 cm), with a pulse length of 100 μs, and an interval between pulses of 100 ms. Once the electroporation generator is started, it provides the programmed pulsed electric field in the pancreatic head lesion through the R.E.D. probe. At this voltage, the regions of the lesion closer to the tips of the cathode and anode undergo irreversible electroporation with permanent permeabilization of the cell membranes, due to the higher field strength at this region. This causes leakage of intracellular components, including tumor antigens and DAMPs, loss of homeostasis and cell death. At this voltage, the regions of the lesion further away from the tips of the cathode and anode, where the field strength is lower, undergo reversible electroporation with transient permeabilization of the cell membranes. Again, this causes leakage of intracellular components including tumor antigens and DAMPs into the extracellular microenvironment, but not permanent cell death. Thirty seconds after the electroporation step is completed, the toll like receptor 9 solution is injected, travels through the perforations and into the lesion to facilitate a targeted inflammatory response to exposed tumor antigens and DAMPs, to generate an adaptive memory immune response.

### Patient Application

The protocol is the same as above, but an electrocardiogram (EKG) device is connected to the patient for monitoring of heart rate and electrocardiac activity during the entire procedure with discharge of pulses between R-wave intervals of the cardiac cycle. This enhanced electroporation therapy can be administered with or without concurrent systemic intravenous immunotherapy infusion, such as but not limited to immune checkpoint inhibitors of CTLA-4, PD-1, and PD-L1.

### Example 2A: Treating Stage 4 Metastatic Disease in the Liver

### Animal Model

The R.E.D. is connected to the corresponding electroporation generator. To eliminate dead air space in the inner cannula, which is in communication with the fluid infusion port and tubing at the proximal end of the device, a syringe containing talimogene laherparepvec (immunotherapy) solution is attached to the luer lock of the fluid infusion port and tubing, and flushed forward until solution is seen dripping from perforations at the distal portion of the inner cannula. The probe is now primed.

The probe is inserted into the liver malignant lesion. The outer sheath of the R.E.D. is unsheathed so that the anode on the tip of the outer sheath is 1-2 cm apart from the cathode at the tip of the inner cannula needle along a linear plane, and perforations along the entire length of the unsheathed inner cannula are exposed and can deliver the immunotherapy solution to the lesion. The generator is programmed to deliver 50 pulses each at 2000V (which corresponds to a field strength of 1000 to 2000V/cm due to the distance between the anode-cathode being 1-2 cm apart), with a pulse length of 100 μs, and an interval between pulses of 100 ms. Once the electroporation generator is started, it provides the programmed pulsed electric field in the right liver lesion through the R.E.D. probe. At this voltage, the regions of the lesion closer to the tips of the cathode and anode undergo irreversible electroporation with permanent permeabilization of the cell membranes due to the higher field strength at this region, causing leakage of intracellular components including tumor antigens and DAMPs, loss of homeostasis and cell death. At this voltage, the regions of the lesion further away from the tips of the cathode and anode undergo reversible electroporation with transient permeabilization of the cell membranes due to lower field strength at this region, causing leakage of intracellular components including tumor antigens and DAMPs into the extracellular microenvironment, but not permanent cell death. Thirty seconds after the electroporation is discharged into the right liver malignant lesion, the talimogene laherparepvec solution is injected, travels through the perforations at the distal end of the exposed inner cannula into the lesion to facilitate a targeted inflammatory response to exposed tumor antigens and DAMPs, and thereby generates an adaptive memory immune response.

### Patient Application

The protocol is the same as above, but an electrocardiogram (EKG) device is connected to the patient for monitoring of heart rate and electrocardiac activity during the entire procedure with discharge of pulses between R-wave intervals of the cardiac cycle. Also, to isolate tumor location, a non-contrast or contrast enhanced computed tomography (CT) scan is performed immediately prior to the procedure to localize metastatic lesions in the body, which in this example are within the right lobe of the liver. And also, the electroporation probe would be inserted into the right liver malignant lesion under percutaneous CT imaging guidance. This enhanced electroporation therapy can be administered with or without concurrent systemic intravenous immunotherapy infusion, such as but not limited to immune checkpoint inhibitors of CTLA-4, PD-1, and PD-L1.

### Example 2B: Treating Stage 4 Melanoma With Metastatic Disease in the Liver

### Animal Model

The same protocol is used as in Example 2A, but with the generator programmed to deliver 1000V (which corresponds to a field strength of 500 to 1000V/cm due to the distance between the anode-cathode being 1 to 2 cm apart), and with 50 pulses each with a pulse length of 100 μs, and an interval between pulses of 100 ms.

### Patient Application

The same differences from Example 2B as set forth in example 2A are used in patients.

### Example 3: Treating Stage 4 Melanoma With Metastatic Disease to the Local Lymph Node

### Animal Model

The R.E.D. is connected to the electroporation generator. To eliminate dead air space in the inner cannula, which is in communication with the fluid infusion port and tubing at the proximal end of the device, a syringe containing interleukin-2 (IL-2) (immunotherapy) solution is attached to the luer lock of the fluid infusion port and tubing, and flushed forward until solution is seen dripping from perforations at the distal portion of the inner cannula. The probe is now primed.

The probe is inserted into the lymph node region. The outer sheath of the R.E.D. is unsheathed so that the anode on the tip of the outer sheath is 1 cm apart from the cathode at the tip of the inner cannula needle along a linear plane, and perforations along the entire length of the unsheathed inner cannula are exposed and can deliver the immunotherapy solution to the lesion. The generator is programmed to deliver 50 pulses each at 500V (which corresponds to a field strength of 500V/cm due to the distance between the anode-cathode being 1-2 cm apart), each with a pulse length of 100 μs, and an interval between pulses of 100 ms. Once the electroporation generator is started, it provides the programmed pulsed electric field in the right supraclavicular lymph node through the R.E.D. probe. At this voltage, the regions of the lesion closer and further away from the tips of the cathode and anode undergo reversible electroporation with transient permeabilization of the cell membranes due to lower field strength, causing leakage of intracellular components including tumor antigens and DAMPs into the extracellular microenvironment, but not permanent cell death. One minute after the electroporation is discharged into the right supraclavicular lymph node, the IL-2 solution in fluid communication with the perforations at the distal end of the exposed inner cannula is injected into the lesion to facilitate a targeted inflammatory response to exposed tumor antigens and DAMPs, to thereby generate an adaptive memory immune response.

### Patient Application

The protocol is the same as above, but an electrocardiogram (EKG) device is connected to the patient for proper monitoring of heart rate and electrocardiac activity during the entire procedure with discharge of pulses between R-wave intervals of the cardiac cycle. Also, the probe is inserted into the right supraclavicular lymph node under percutaneous CT imaging guidance. This enhanced electroporation therapy can be administered with or without concurrent systemic intravenous immunotherapy infusion, such as but not limited to immune checkpoint inhibitors of CTLA-4, PD-1, and PD-L1.

### Example 4: Treating Stage 4 Melanoma With Metastatic Disease to the Bones

### Animal Model

The R.E.D. is connected to the corresponding electroporation generator. To eliminate dead air space in the inner cannula, which is in communication with the fluid infusion port and tubing at the proximal end of the device, a syringe containing attenuated Staphylococcus aureus (immunotherapy) solution is attached to the luer lock of the fluid infusion port and tubing, and flushed forward until solution is seen dripping from perforations at the distal portion of the inner cannula. The probe is now primed.

The probe is inserted into the L1 vertebral body malignant lesion. The outer sheath of the R.E.D. is unsheathed so that the anode on the tip of the outer sheath is 0.5 cm apart from the cathode at the tip of the inner cannula needle along a linear plane, and perforations along the entire length of the unsheathed inner cannula are exposed and can deliver the immunotherapy solution to the lesion. The generator is programmed to deliver 75 pulses each at 1000V (which corresponds to a field strength of 2000V/cm due to the distance between the anode-cathode being 0.5 cm), each with a pulse length of 100 μs, and an interval between pulses of 100 ms. Once the electroporation generator is started, it provides the programmed pulsed electric field in the L1 vertebral body lesion through the R.E.D. probe. At this voltage, the regions of the lesion closer to the tips of the cathode and anode undergo irreversible electroporation with permanent permeabilization of the cell membranes due to the higher field strength at this region, causing leakage of intracellular components including tumor antigens and DAMPs, loss of homeostasis and cell death. The regions of the lesion further away from the tips of the cathode and anode undergo reversible electroporation with transient permeabilization of the cell membranes due to lower field strength at this region, causing leakage of intracellular components including tumor antigens and DAMPs into the extracellular microenvironment, but not permanent cell death. 1 minute after the electroporation is discharged into the L1 vertebral body malignant lesion, the attenuated Staphylococcus aureus solution in fluid communication with the perforations at the distal end of the exposed inner cannula is injected into the lesion to facilitate a targeted inflammatory response to exposed tumor antigens and DAMPs, to generate an adaptive memory immune response.

### Patient Application

The protocol is the same as above, but an electrocardiogram (EKG) device is connected to the patient for proper monitoring of heart rate and electrocardiac activity during the entire procedure with discharge of pulses between R-wave intervals of the cardiac cycle. A non-contrast or contrast enhanced computed tomography (CT) scan is performed immediately prior to the procedure to localize metastatic lesions in the body, which in this example is seen within the L1 vertebral body. Also, the probe is inserted into the L1 vertebral body malignant lesion under percutaneous CT or fluoroscopic imaging guidance. This enhanced electroporation therapy can be administered with or without concurrent systemic intravenous immunotherapy infusion such as but not limited to immune checkpoint inhibitors of CTLA-4, PD-1, and PD-L1.

### Example 5: Treating Stage 3B Hepatocellular Carcinoma

### Animal Model

The R.E.D. is connected to the corresponding electroporation generator. To eliminate dead air space in the inner cannula, which is in communication with the fluid infusion port and tubing at the proximal end of the device, a syringe containing toll like receptor 7 (immunotherapy) solution is attached to the luer lock of the fluid infusion port and tubing, and flushed forward until solution is seen dripping from perforations at the distal portion of the inner cannula. The probe is now primed.

The probe is inserted into the left liver malignant lesion. The outer sheath of the R.E.D. is unsheathed so that the anode on the tip of the outer sheath is 1 cm apart from the cathode at the tip of the inner cannula needle along a linear plane, and perforations along the entire length of the unsheathed inner cannula are exposed and can deliver the immunotherapy solution to the lesion. The generator is programmed to deliver 75 pulses each at 2000V (which corresponds to a field strength of 2000V/cm due to the distance between the anode-cathode being 1 cm), each with a pulse length of 100 μs, and an interval between pulses of 100 ms. Once the electroporation generator is started, it provides the programmed pulsed electric field in the left liver lesion through the R.E.D. probe. At this voltage, the regions of the lesion closer to the tips of the cathode and anode undergo irreversible electroporation with permanent permeabilization of the cell membranes due to the higher field strength at this region, causing leakage of intracellular components including tumor antigens and DAMPs, loss of homeostasis and cell death. At this voltage, the regions of the lesion further away from the tips of the cathode and anode undergo reversible electroporation with transient permeabilization of the cell membranes due to lower field strength at this region, causing leakage of intracellular components including tumor antigens and DAMPs into the extracellular microenvironment, but not permanent cell death. Thirty seconds after the electroporation is discharged into the left liver lesion, the toll like receptor 7 solution in fluid communication with the perforations at the distal end of the exposed inner cannula is injected into the lesion to facilitate a targeted inflammatory response to exposed tumor antigens and DAMPs, to thereby generate an adaptive memory immune response. The R.E.D. probe is then pulled back from the skin, and electroporation is performed again with the same parameters, and toll like receptor 7 solution injected again afterwards into a more proximal portion of the malignant left liver lesion.

### Patient Application

The protocol is the same as above, but an electrocardiogram (EKG) device is connected to the patient for proper monitoring of heart rate and electrocardiac activity during the entire procedure with discharge of pulses between R-wave intervals of the cardiac cycle. Also, a non-contrast or contrast enhanced computed tomography (CT) scan is performed immediately prior to the procedure to localize the malignant lesion at the left lobe of the liver. Also, the probe is inserted into the left liver malignant lesion under percutaneous CT imaging guidance, and when the probe is pulled back after the first combined electroporation application, it is pulled back by about 1 cm. This enhanced electroporation therapy can be administered with or without concurrent systemic intravenous immunotherapy infusion such as but not limited to immune checkpoint inhibitors of CTLA-4, PD-1, and PD-L1.

### Example 6: Treating Stage 4 Colorectal Cancer With Metastasis to the Lungs

### Animal Model

The R.E.D. is connected to the electroporation generator. To eliminate dead air space in the inner cannula, which is in communication with the fluid infusion port and tubing at the proximal end of the device, a syringe containing a hydrogel and/or cryogel formulation of interferon-alpha (IFN-alpha) (immunotherapy) solution is attached to the luer lock of the fluid infusion port and tubing, and flushed forward until solution is seen dripping from perforations at the distal portion of the inner cannula. The probe is now primed.

The probe is inserted into the right upper lobe lung malignant lesion. The outer sheath of the R.E.D. is unsheathed so that the anode on the tip of the outer sheath is 1 cm apart from the cathode at the tip of the inner cannula needle along a linear plane, and perforations along the entire length of the unsheathed inner cannula are exposed and can deliver the hydrogel and/or cryogel immunotherapy solution to the lesion. The generator is programmed to deliver 50 pulses each at 2000V (which corresponds to a field strength of 2000 V/cm due to the distance between the anode-cathode being 1 cm), each with a pulse length of 100 μs, and an interval between pulses of 100 ms. Once the electroporation generator is started, it provides the programmed pulsed electric field in the left liver lesion through the R.E.D. probe. At this voltage, the regions of the lesion closer to the tips of the cathode and anode undergo irreversible electroporation with permanent permeabilization of the cell membranes due to the higher field strength at this region, causing leakage of intracellular components including tumor antigens and DAMPs, loss of homeostasis and cell death. Regions of the lesion further away from the tips of the cathode and anode undergo reversible electroporation with transient permeabilization of the cell membranes due to lower field strength at this region, causing leakage of intracellular components including tumor antigens and DAMPs into the extracellular microenvironment, but not permanent cell death. Thirty seconds after the electroporation is discharged into the right upper lobe lung malignant lesion, the IFN-alpha solution in fluid communication with the perforations at the distal end of the exposed inner cannula is injected into the lesion to facilitate a targeted inflammatory response to exposed tumor antigens and DAMPs, to generate an adaptive memory immune response. The R.E.D. probe is then pulled back from the skin, and electroporation is performed again with the same parameters, and IFN-alpha solution injected again afterwards in a similar fashion to a more proximal portion of the malignant right upper lobe lung malignant lesion.

### Patient Application

The protocol is the same as above, but an electrocardiogram (EKG) device is connected to the patient for proper monitoring of heart rate and electrocardiac activity during the entire procedure with discharge of pulses between R-wave intervals of the cardiac cycle. A non-contrast or contrast enhanced computed tomography (CT) scan is performed immediately prior to the procedure to localize the malignant lesion at the right upper lobe of the lung. The probe is inserted into the right upper lobe lung malignant lesion under percutaneous CT imaging guidance. When the probe is pulled back after the first combined electroporation application, it is pulled back by about 1 cm. This enhanced electroporation therapy can be administered with or without concurrent systemic intravenous immunotherapy infusion such as but not limited to immune checkpoint inhibitors of CTLA-4, PD-1, and PD-L1.

### Example 7: Treating Stage 3B Prostate Cancer

### Animal Model

The R.E.D. is connected to the corresponding electroporation generator. To eliminate dead air space in the inner cannula, which is in communication with the fluid infusion port and tubing at the proximal end of the device, a syringe containing bacille Calmette-Guerin (BCG) (immunotherapy) solution is attached to the luer lock of the fluid infusion port and tubing, and flushed forward until solution is seen dripping from perforations at the distal portion of the inner cannula. The probe is now primed.

The probe is inserted into the left liver malignant lesion. The outer sheath of the R.E.D. is unsheathed so that the anode on the tip of the outer sheath is 1 cm from the cathode at the tip of the inner cannula needle along a linear plane, and perforations along the entire length of the unsheathed inner cannula are exposed and can deliver the immunotherapy solution to the lesion. The generator is programmed to deliver 100 pulses each at 2000V (which corresponds to a field strength of 2000V/cm due to the distance between the anode-cathode being 1 cm), each with a pulse length of 100 μs, and an interval between pulses of 100 ms. Once the electroporation generator is started, it provides the programmed pulsed electric field in the prostate gland lesion through the R.E.D. probe. At this voltage, the regions of the lesion closer to the tips of the cathode and anode undergo irreversible electroporation with permanent permeabilization of the cell membranes due to the higher field strength at this region, causing leakage of intracellular components including tumor antigens and DAMPs, loss of homeostasis and cell death. The regions of the lesion further away from the tips of the cathode and anode undergo reversible electroporation with transient permeabilization of the cell membranes due to lower field strength at this region, causing leakage of intracellular components including tumor antigens and DAMPs into the extracellular microenvironment, but not permanent cell death. Thirty seconds after the electroporation is discharged into the prostate gland lesion, the BCG solution in fluid communication with the perforations at the distal end of the exposed inner cannula is injected into the lesion to facilitate a targeted inflammatory response to exposed tumor antigens and DAMPs to generate an adaptive memory immune response. The R.E.D. probe is then pulled back from the skin, and electroporation is performed again with the same parameters, and BCG solution injected again afterwards in a similar fashion to a more proximal portion of the malignant prostate gland lesion.

### Patient Application

The protocol is the same as above, but an electrocardiogram (EKG) device is connected to the patient for proper monitoring of heart rate and electrocardiac activity during the entire procedure with discharge of pulses between R-wave intervals of the cardiac cycle. Also, a non-contrast or contrast enhanced computed tomography (CT) scan is performed immediately prior to the procedure to localize the malignant lesion at the prostate gland. Further, the probe is inserted into the left liver malignant lesion under percutaneous under CT imaging guidance. When the probe is pulled back after the first combined electroporation application, it is pulled back by about 1 cm. This enhanced electroporation therapy can be administered with or without concurrent systemic intravenous immunotherapy infusion such as but not limited to immune checkpoint inhibitors of CTLA-4, PD-1, and PD-L1.

### Example 8: Treating Venous Malformation

The R.E.D. is connected to the electroporation generator. To eliminate dead air space in the inner cannula, which is in communication with the fluid infusion port and tubing at the proximal end of the device, a syringe containing bleomycin solution is attached to the luer lock of the fluid infusion port and tubing, and flushed forward until solution is seen dripping from perforations at the distal portion of the inner cannula. The probe is now primed.

The probe is inserted into a venous malformation. The outer sheath of the R.E.D. is unsheathed so that the anode on the tip of the outer sheath is 1 cm from the cathode at the tip of the inner cannula needle along a linear plane, and perforations along the entire length of the unsheathed inner cannula are exposed and can deliver the bleomycin into the venous malformation. The generator is programmed to deliver 100 pulses each at 400 V (which corresponds to a field strength of 400 V/cm due to the distance between the anode-cathode being 1 cm), each with a pulse length of 100 μs, and an interval between pulses of 100 ms. Once the electroporation generator is started, it provides the programmed pulsed electric field in the venous malformation through the R.E.D. probe. Thirty seconds after the electroporation is discharged into the venous malformation, the bleomycin solution in fluid communication with the perforations at the distal end of the exposed inner cannula is injected into the venous malformation.

### Patient Application

The protocol is the same as above, but an electrocardiogram (EKG) device is connected to the patient for proper monitoring of heart rate and electrocardiac activity during the entire procedure with discharge of pulses between R-wave intervals of the cardiac cycle.

### Example 9: Treating Arterio-Venous Malformation

The R.E.D. is connected to the electroporation generator. To eliminate dead air space in the inner cannula, which is in communication with the fluid infusion port and tubing at the proximal end of the device, a syringe containing bleomycin solution is attached to the luer lock of the fluid infusion port and tubing, and flushed forward until solution is seen dripping from perforations at the distal portion of the inner cannula. The probe is now primed.

The probe is inserted into soft tissues adjacent to an arterio-venous malformation. The outer sheath of the R.E.D. is unsheathed so that the anode on the tip of the outer sheath is 0.5 cm from the cathode at the tip of the inner cannula needle along a linear plane, and perforations along the entire length of the unsheathed inner cannula are exposed and can deliver the bleomycin into the venous malformation. The generator is programmed to deliver 100 pulses each at 1000 V (which corresponds to a field strength of 500 V/cm due to the distance between the anode-cathode being 0.5 cm), each with a pulse length of 100 μs, and an interval between pulses of 100 ms. Once the electroporation generator is started, it provides the programmed pulsed electric field in the venous malformation through the R.E.D. probe. Thirty seconds after the electroporation is discharged into the soft tissues adjacent to the arterio-venous malformation, the bleomycin solution in fluid communication with the perforations at the distal end of the exposed inner cannula is injected into the soft tissues adjacent to the arterio-venous malformation.

### Patient Application

The protocol is the same as above, but an electrocardiogram (EKG) device is connected to the patient for proper monitoring of heart rate and electrocardiac activity during the entire procedure with discharge of pulses between R-wave intervals of the cardiac cycle.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also noted that as used herein and in the appended claims, the singular forms “a,” “an,” and “the” include plural reference, and the plural include singular forms, unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, including but not limited to Variant Sequences, and that such modifications and variations are considered to be within the scope of this invention as defined by the appended claims.

What is claimed is:

1. An electroporation probe, comprising:
a shaft which is a metallic trocar and is electrically connected to a cathode terminal of a power source, said shaft having a proximal end, a distal end, the distal end of the shaft further including a tip;
said shaft is surrounded by a polyimide tube having multiple perforations in a wall of the tube, said tube having an open proximal end and a closed distal end, the distal end of the tube being attached in a leakproof manner with the outer surface of the shaft, wherein a lumen is formed by a space between the inner surface of the tube and the outer surface of the shaft, and the lumen accesses a source of injectable fluid;
a tubular movable sheath covering the tube and being slidable longitudinally over the outer surface of the tube between a first position covering and sealing one or more of said perforations and a second position unsealing all of said perforations, said sheath having a proximal end, a distal end, and including a conductive anode portion towards said distal end, said anode portion being electrically connected to an anode terminal of said power source, said anode portion not being in direct electrical contact with said tube or the shaft;
a probe holder having an interior channel and a sliding tab, said sheath, said shaft and said tube extending through the interior channel, and said sliding tab being attached to the sheath such that a longitudinal movement of the sliding tab moves the sheath between the first position and the second position, wherein:
length of said anode portion is greater than the separation between a distal end of the anode portion and the distal end of sheath, and
separation between the distal end of the polyimide tube and the distal end of said anode portion, in said first position, is 7.5 to up to but less than 30 mm, and
said injectable fluid is an immunotherapy or an anti-cancer agent.

2. The probe of claim 1 wherein, said distal end of the shaft further includes a tapered tip.

3. The probe of claim 1 wherein said power source is a DC power source, said DC power source is one of:
i) a DC battery, and
ii) a DC output generated from an AC supply.

4. The probe of claim 1 wherein said anode portion is electrically connected to the anode terminal through a first electrical conductor included in the movable sheath.

5. The probe of claim 1 wherein said immunotherapy agent is one of a hydrogel or a cryogel, either of the hydrogel or the cryogel being impregnated with one or more of medicinal drugs for treating tissues.

6. The probe of claim 1, wherein said shaft is flexible.

7. An electroporation probe comprising:
a shaft which is a metallic trocar and is electrically connected to a cathode terminal of a power source, said shaft having a proximal end, a distal end, the distal end of the shaft further including a tip;
said shaft is surrounded by a polyimide tube having multiple perforations in a wall of the tube, said tube having an open proximal end and a closed distal end, the distal end of the tube being attached in a leakproof manner with the outer surface of the shaft, wherein a lumen is formed by a space between the inner surface of the tube and the outer surface of the shaft, and the lumen accesses a source of injectable fluid;
a tubular movable sheath covering the tube and being slidable longitudinally over the outer surface of the tube between a first position covering and sealing one or more of said perforations and a second position unsealing all of said perforations, said sheath having a proximal end, a distal end, said sheath further including a conductive anode portion towards said distal end, said anode portion being electrically connected to an anode terminal of said power source, said anode portion not being in direct electrical contact with said tube or the shaft;
a probe holder having an interior channel and a sliding tab, said sheath, said shaft and said tube extending through the interior channel, and said sliding tab being attached to the sheath such that a longitudinal movement of the sliding tab moves the sheath between the first position and the second position, wherein:
length of said anode portion is greater than the separation between a distal end of the anode portion and the distal end of sheath, and
separation between the distal end of the polyimide tube and the distal end of said anode portion, in said second position, is 27.5 to 30 mm, and
said injectable fluid is an immunotherapy or an anti-cancer agent.

8. The probe of claim 7 wherein, said distal end of the shaft further includes a tapered tip.

9. The probe of claim 7 wherein said power source is a DC power source, said DC power source is one of:
i) a DC battery, and
ii) a DC output generated from an AC supply.

10. The probe of claim 7 wherein said anode portion is electrically connected to the anode terminal through a first electrical conductor included in the movable sheath.

11. The probe of claim 7 wherein said immunotherapy agent is one of a hydrogel or a cryogel, either of the hydrogel or the cryogel being impregnated with one or more of medicinal drugs for treating tissues.

12. The probe of claim 7, wherein said shaft is flexible.

13. An electroporation probe comprising:
a shaft having a proximal end, a distal end, the distal end of the shaft further including a metallic tip, said tip being electrically connected to a cathode terminal of a power source;
said shaft is surrounded by a polyimide tube having multiple perforations in a wall of the tube, said tube having an open proximal end and a closed distal end, the distal end of the tube being attached in a leakproof manner with the outer surface of the shaft, wherein a lumen is formed by a space between the inner surface of the tube and the outer surface of the shaft, and the lumen accesses a source of injectable fluid;
a tubular movable sheath covering the tube and being slidable longitudinally over the outer surface of the tube between a first position covering and sealing one or more of said perforations and a second position unsealing all of said perforations, said sheath having a proximal end, a distal end, said sheath further including a conductive anode portion towards said distal end, said anode portion being electrically connected to an anode terminal of said power source, said anode portion not being in direct electrical contact with said tube or the shaft;
a probe holder having an interior channel and a sliding tab, said sheath, said shaft and said tube extending through the interior channel, and said sliding tab being attached to the sheath such that a longitudinal movement of the sliding tab moves the sheath between the first position and the second position, wherein:
length of said anode portion is greater than the separation between a distal end of the anode portion and the distal end of sheath, and
separation between the distal end of the polyimide tube and the distal end of said anode portion, in said first position, is 7.5 to up to but less than 30 mm, and
said injectable fluid is an immunotherapy or an anti-cancer agent.

14. The probe of claim 13 wherein said power source is a DC power source, said DC power source is one of:
i) a DC battery, and
ii) a DC output generated from an AC supply.

15. The probe of claim 13 wherein said anode portion is electrically connected to the anode terminal through a first electrical conductor included in the movable sheath.

16. The probe of claim 13 wherein said immunotherapy agent is one of a hydrogel or a cryogel, either of the hydrogel or the cryogel being impregnated with one or more of medicinal drugs for treating tissues.

17. The probe of claim 13, wherein said shaft is flexible.

18. An electroporation probe comprising:
a shaft having a proximal end, a distal end, the distal end of the shaft further including a metallic tip, said tip being electrically connected to a cathode terminal of a power source;
said shaft is surrounded by a polyimide tube having multiple perforations in a wall of the tube, said tube having an open proximal end and a closed distal end, the distal end of the tube being attached in a leakproof manner with the outer surface of the shaft, wherein a lumen is formed by a space between the inner surface of the tube and the outer surface of the shaft, and the lumen accesses a source of injectable fluid;
a tubular movable sheath covering the tube and being slidable longitudinally over the outer surface of the tube between a first position covering and sealing one or more of said perforations and a second position unsealing all of said perforations, said sheath having a proximal end, a distal end, said sheath further including a conductive anode portion towards said distal end, said anode portion being electrically connected to an anode terminal of said power source, said anode portion not being in direct electrical contact with said tube or the shaft;
a probe holder having an interior channel and a sliding tab, said sheath, said shaft and said tube extending through the interior channel, and said sliding tab being attached to the sheath such that a longitudinal movement of the sliding tab moves the sheath between the first position and the second position, wherein:
length of said anode portion is greater than the separation between a distal end of the anode portion and the distal end of sheath, and
separation between the distal end of the polyimide tube and the distal end of said anode portion, in said second position, is 27.5 to 30 mm, and
said injectable fluid is an immunotherapy or an anti-cancer agent.

19. The probe of claim 18 wherein said power source is a DC power source, said DC power source is one of:
i) a DC battery, and
ii) a DC output generated from an AC supply.

20. The probe of claim 18 wherein said anode portion is electrically connected to the anode terminal through a first electrical conductor included in the movable sheath.

21. The probe of claim 18 wherein said immunotherapy agent is one of a hydrogel or a cryogel, either of the hydrogel or the cryogel being impregnated with one or more of medicinal drugs for treating tissues.

22. The probe of claim 18, wherein said shaft is flexible.

23. An electroporation probe comprising:
a shaft having a proximal end, a distal end, a lumen, and multiple perforations, the distal end of the shaft further including a metallic tip, said tip being electrically connected to a cathode terminal of a power source;
said lumen extending longitudinally through the shaft, and having an open proximal end and a closed distal end, said open proximal end of the lumen access a source of injectable fluid, and said perforations extend through the walls of the shaft and connect the exterior of the shaft with the lumen;
a tubular movable sheath covering the shaft and being slidable longitudinally over the outer surface of the shaft between a first position covering and sealing one or more of said perforations and a second position unsealing all of said perforations, said sheath having a proximal end, a distal end, said sheath further including a conductive anode portion towards said distal end, said anode portion being electrically connected to an anode terminal of said power source, said anode portion not being in direct electrical contact with the tip;
a probe holder having an interior channel and a sliding tab, said sheath, and said shaft extending through the interior channel, and said sliding tab being attached to the sheath such that a longitudinal movement of the sliding tab moves the sheath between the first position and the second position, wherein:
length of said anode portion is greater than the separation between a distal end of the anode portion and the distal end of sheath, and
separation between the distal end of the sheath and the distal end of said anode portion, in said first position, is 7.5 to 10 mm, and
said injectable fluid is an immunotherapy or an anti-cancer agent.

24. The probe of claim 23 wherein said power source is a DC power source, said DC power source is one of:
i) a DC battery, and
ii) a DC output generated from an AC supply.

25. The probe of claim 23 wherein said anode portion is electrically connected to the anode terminal through a first electrical conductor included in the movable sheath.

26. The probe of claim 23 wherein said immunotherapy agent is one of a hydrogel or a cryogel, either of the hydrogel or the cryogel being impregnated with one or more of medicinal drugs for treating tissues.

27. The probe of claim 23, wherein said shaft is flexible.

28. An electroporation probe comprising:
a shaft which is a metallic trocar and is electrically connected to a cathode terminal of a power source, said shaft having a proximal end, a distal end, a lumen, and multiple perforations, the distal end of the shaft further including a tip;
said lumen extending longitudinally through the shaft, and having an open proximal end and a closed distal end, said open proximal end of the lumen access a source of injectable fluid, and said perforations extend through the walls of the shaft and connect the exterior of the shaft with the lumen;
a tubular movable sheath covering the shaft and being slidable longitudinally over the outer surface of the tube between a first position covering and sealing one or more of said perforations and a second position unsealing all of said perforations, said sheath having a proximal end, a distal end, said sheath further including a conductive anode portion towards said distal end, said anode portion being electrically connected to an anode terminal of said power source, said anode portion not being in direct electrical contact with the shaft;
a probe holder having an interior channel and a sliding tab, said sheath and said shaft extending through the interior channel, and said sliding tab being attached to the sheath such that a longitudinal movement of the sliding tab moves the sheath between the first position and the second position, wherein:
length of said anode portion is greater than the separation between a distal end of the anode portion and the distal end of sheath,
separation between the distal end of the sheath and the distal end of said anode portion, in said first position, is 7.5 to 10 mm, and
said injectable fluid is an immunotherapy or an anti-cancer agent.

29. The probe of claim 28 wherein, said distal end of the shaft further includes a tapered tip.

30. The probe of claim 28 wherein said power source is a DC power source, said DC power source is one of:
i) a DC battery, and
ii) a DC output generated from an AC supply.

31. The probe of claim 28 wherein said anode portion is electrically connected to the anode terminal through a first electrical conductor included in the movable sheath.

32. The probe of claim 28 wherein said immunotherapy agent is one of a hydrogel or a cryogel, either of the hydrogel or the cryogel being impregnated with one or more of medicinal drugs for treating tissues.

33. The probe of claim 28, wherein said shaft is flexible.
